(19) United States
(12) Patent Application Publication
(10) Pub. No.: US 2021/0395691 A1
(43) Pub. Date: Dec. 23, 2021

(54) MEANS AND METHODS OF PREVENTING OR REVERSING AGING

(71) Applicant: Figene, LLC, Huoston, TX (US)

(72) Inventors: Pete O'Heeron, Huoston, TX (US); Thomas Ichim, San Diego, CA (US)

(21) Appl. No.: 17/309,208

(22) PCT Filed: Nov. 8, 2019
(86) PCT No.: PCT/US2019/060446

§ 371 Date: May 6, 2021

Related U.S. Application Data
(60) Provisional application No. 62/758,240, filed on Nov. 9, 2018.

(51) Int. Cl. C12N 5/077; C12M 1/26

(52) U.S. Cl. CPC C12N 5/0656; C12M 33/14; C12N 2501/13; C12N 2501/115; C12N 2501/24; C12N 2500/38; C12N 2501/998; C12N 2501/2301; C12N 2501/999

(57) ABSTRACT

The present disclosure provides means of regenerating/restoring aged and/or damaged tissue or treating a medical condition by providing an extracorporeal circuit containing cells, including fibroblasts or dedifferentiated fibroblasts. that are in contact with plasma but not blood cells extracorporeally in a manner allowing for exchange of factors between a subject and an extracorporeally residing mass of cells. The disclosure provides means of titrating factors produced by the extracorporeal regenerative cell mass, thus allowing for a regenerative effect.

This application claims priority to U.S. Provisional Patent Application Ser. No. 62/758,240, filed Nov. 9, 2018, which is incorporated by reference herein in its entirety.

### TECHNICAL FIELD

Embodiments of the disclosure concern at least the fields of cell biology, molecular, biology, immunology, and medicine.

### BACKGROUND

Previous studies have demonstrated that connection of circulation between young animals and old animals results in a systemic anti-aging effect. For example, one study examined the influence of systemic factors on aged progenitor cells from hepatic tissues, by establishing a shared circulatory system between young and old mice (heterochronic parabiosis), thus exposing old mice to factors present in young serum. Notably, this pairing restored the activation of Notch signaling as well as the proliferation and regenerative capacity of aged satellite cells. In addition, the exposure of satellite cells from old mice to young serum enhanced the expression of the Notch ligand (Delta), increased Notch activation, and enhanced proliferation in vitro. Furthermore, heterochronic parabiosis increased aged hepatocyte proliferation and restored the cEBP-alpha complex to levels seen in young animals. These results suggest that the age-related decline of progenitor cell activity can be modulated by systemic factors that change with age [1]. Another study found young blood contained factors that induce vascular remodeling, culminating in increased neurogenesis and improved olfactory discrimination in aging mice using similar heterochronic parabiosis models. Identification of molecular factors associated with improvement of cerebral vascularity and neurogenesis revealed a role for the molecule GDF11 [2].

Unfortunately, means of connecting human circulations is extremely difficult and unethical. Thus, there is a need in the art for supplying regenerative factors to a subject in need thereof in a practical manner.

### BRIEF SUMMARY

The present disclosure is directed to systems, methods, and compositions to provide one or more regenerative factors to one or more subjects in need. Aspects of the disclosure provide for systems, compositions, and methods in which the circulatory system of a subject having a medical condition and/or that is aged is exposed to one or more beneficial regenerative factors produced from particular cells, wherein the regenerative factor(s) were produced upon exposure of the cells to the blood or plasma from the subject. In other embodiments subject is exposed to the system to prevent or delay the onset of one or more symptoms of aging. In specific embodiments, cells that are in-line in a system with a subject produce one or more regenerative factors upon exposure to one or more degenerative factors that come from the circulation of the subject, and the one or more regenerative factors are provided to the subject in-line in the system. Such regenerative factors then ameliorate at least one symptom of the medical condition and/or reduce at least partially one or more effects of aging.

In particular embodiments, the disclosure utilizes an extracorporeal circuit to provide one or more regenerative factors to one or more subjects in need. In specific embodiments, the extracorporeal circuit comprises tubing linking at least one subject and at least one bioreactor. In specific embodiments, the disclosure encompasses a circuit that links a subject and a bioreactor, in which one or more certain factors are shared between the subject and the bioreactor. In specific embodiments, the disclosure separates one or more cellular components of the subject from one or more cellular components of one or more bioreactors. In particular embodiments, this separation occurs by employing at least one semi-permeable membrane in a location in the tubing (in specific embodiments, unless a hollow fiber is utilized), on the subject, and/or on the bioreactor. In some embodiments, the bioreactor comprises cells that secrete one or more factors that are useful to the subject. In some embodiments, the bioreactor detects one or more factors that are from the subject. In specific embodiments, the bioreactor secretes factors at a rate dependent on the rate of factors detected from the subject.

In some embodiments, the subject in need is an individual that has at least one medical condition requiring therapy and/or is of an age that requires a need for a replenishment of one or more factors and/or in need for a reduction of at least one or more harmful factors. In at least some cases, the subject is an individual with abnormal levels of factors associated with aging. In other cases, the subject is a individual with chronic or acute abnormal levels of degenerative factors, for example. In some embodiments, the subject is an organ. In at least some cases, the organ is from an individual with abnormal levels of one or more degenerative factors and/or one or more factors associated with aging. In other cases, the organ is from a donor, wherein the regenerative factors supplied by the bioreactor(s) are useful in keeping the organ fully viable; the organ may be in need of being used for a transplant, for example. In particular embodiments, the subject is one or more than one tissues.

Embodiments of the disclosure provide means of generating and/or utilizing cells in a bioreactor. In some embodiments of the disclosure, the cells generated and/or utilized are from a young subject or from a subject that lacks one or more signs of aging. In particular embodiments of the disclosure, the cells generated and/or utilized have one or more cell surface molecules. In particular embodiments, the cells generated and/or utilized lack one or more particular cell surface molecules. In particular embodiments of the disclosure the cells generated and/or utilized are cultured using a method that enhances the ability of the cells to secrete one or more regenerative factors and/or to detect or react to one or more degenerative factors. In at least some cases, the cells generated and/or utilized are further manipulated, for example in culture using one or more viral and/or one or more non-viral methods.

Embodiments of the disclosure include systems for treating a subject, wherein the system comprises at least one bioreactor comprising cells, at least one selectively permeable membrane, and tubing connecting at least one of the bioreactor(s), at least one of the selectively permeable membrane(s), and the subject in order to circulate fluid from the subject to the bioreactor, and in specific embodiments with the proviso that if the tubing comprises a hollow fiber then a membrane is optional. The system may further comprise blood or plasma from the subject or comprising organ preservation solution. The cells may be regenerative cells that allow regeneration of cells or tissue, and in specific cases, the regenerative cells are fibroblasts, dedifferentiated fibroblasts, inducible pluripotent cells, parthenogenic derived cells, mesenchymal stem cells, or hematopoietic stem cells, although the stem cells may be of any kind. The regenerative cells may secrete one or more regenerative factors at a basal or at an inducible rate. Examples of regenerative factors include one or more factors selected from the group consisting of AKT, BAMBI, BCL-2, BCL-2XL, BDNF, BIRC5 CDA, CXCR4, dominant negative CCL2, EGF, exosomes, FGF-2, GATA-4 GDF-11, GDNF, hCG, HGF, HIF-1alpha, HLA-G, HO-1, hTERT, IFN-b, IGF-1, IFT-1, LIGHT, miR-126, NK4, NUR77, OCT-4, PGE-1, SDF-1, STC-1, TERT, TRAIL, VEGF, WNT11, XIAP, and a combination thereof. The regenerative cells may produce one or more regenerative factors in response to one or more degenerative factors from the blood or plasma of the subject. In some cases, the regenerative cells secrete regenerative factors at a rate set by the detection of degenerative factors. The rate of regenerative factor secretion from the cells may be a ratio to the rate of degenerative factor detection by the cells. In specific embodiments, the ratio of regenerative factor secretion rate to degenerative factor detection rate is selected from the group consisting of 50:1, 25:1, 10:1, 5:1, 1:1, 1:5, 1:10, 1:25, 1:50, and any ratio therebetween.

In specific embodiments of the disclosure, a selectively permeable membrane is positioned in the tubing, for example between the bioreactor and the subject; the tubing may be comprised of a selectively permeable membrane; and/or the bioreactor may be comprised, at least in part, of a selectively permeable membrane. In some cases, the selectively permeable membrane inhibits or reduces the passage of cellular material (whole cells or fragments thereof) between the subject and a bioreactor. One or more selectively permeable membrane(s) may permit the passage of one or more regenerative and one or more degenerative factors.

The system may be applied to any subject, such as a living animal, or the subject may be an organ (any part of the organ or any part of an organ system) or tissue from an animal; the organ or tissue may come from a donor. Examples of organs include one or more selected from the group consisting of liver, pancreas, gallbladder, stomach, small intestine, large intestine, lung, kidney, heart, spleen, brain, eye, and a combination thereof.

In cases wherein the subject comprises an organ or tissue, the system may also comprise a container connected to at least one of the bioreactor(s) and at least one of the selectively permeable membrane(s) for encapsulating the subject. In specific embodiments, the subject is an organ or tissue and the container is suitable for holding the subject to allow for transfer of organ preservation or other fluid into the tubing.

Embodiments of the disclosure include an extracorporeal method of producing regenerative factors, comprising the step of subjecting a subject to a system encompassed by the disclosure under conditions that allow for secretion of one or more regenerative factors from the cells in the system. The cells may be fibroblasts that are derived from tissues comprising skin, heart, blood vessels, bone marrow, skeletal muscle, liver, pancreas, brain, adipose tissue, placenta, and/or foreskin. The fibroblasts may have one or more surface markers selected from the group consisting of CD73, CD90, CD56, SSEA3, SSEA4, Tra-1-60, Tra-1-81, Tra-2-54, HLA class I, CD13, CD44, CD49b, CD105, aminopeptidase N, hyaluronic acid-binding receptor, collagen/laminin-binding integrin alpha 2, OCT4, NANOG, SOX-2, and a combination thereof. In specific embodiments, the fibroblasts lack one or more surface markers selected from the group consisting of CD14, CD34, CD45, HLA Class II, and a combination thereof.

In particular embodiments of the method, the method comprises the step(s) of culturing fibroblasts in an undifferentiated state for 1, 2, 3, 4, 5, 6, 7, 8, 9, 10, 11, 12, 13, 14, 15, 16, 17, 18, 19, 20, 21, 22, 23, 24, 25 or more days; culturing the fibroblasts from step (a) in the presence of one or more factors selected from the group consisting of nerve growth factor, bFGF, dibutryl cAMP, IBMX, retinoic acid, exendin-4, and a combination thereof; and activating the fibroblasts. Activating the fibroblasts may comprise exposing the fibroblasts to one or more cytokines in cell culture media. Examples of cytokines are one or more selected from the group consisting of IL-1, IFNgamma, and a combination thereof. The cytokines may be at a particular concentration; for example, the concentration of IL-1 may be 1-100 ng/mL, 5-100 ng/mL, 10-100 ng/mL, or 20-40 ng/mL; the concentration of IL-1 may be 1 ng/mL, 5 ng/mL, 10 ng/mL, 20 ng/mL, 30 ng/mL, 40 ng/mL, 50 ng/mL, 60 ng/mL, 70 ng/mL, 80 ng/mL, 90 ng/mL, or 100 ng/mL; the concentration of IFNgamma may be 1-1000 IU, 5-1000 IU, 10-1000 IU, 1-500 IU, 5-500 IU, 10-500 IU, 100-500 IU, or 250 IU; the concentration of IFNgamma may be 1 IU, 5 IU, 10 IU, 50 IU, 100 IU, 200 IU, 250 IU, 300 IU, 400 IU, 500 IU, 600 IU, 700 IU, 800 IU, 900 IU, or 1000 IU. Such concentrations are examples only.

In particular embodiments of the disclosure, exposing the fibroblasts to one or more cytokines induces an increase in the expression of one or more complement inhibitory molecules from the fibroblasts. Examples of complement inhibitory molecules are selected from the group consisting of CD35, CD46, C4BP, CD55, Factor H, and a combination thereof. Activating the fibroblasts may comprise transfecting the fibroblasts with one or more viral and/or non-viral expression systems to induce the expression of one or more regenerative factors. In specific embodiments, the regenerative factors are selected from the group consisting of AKT, BAMBI, BCL-2, BCL-2XL, BDN, BIRC5 CDA, CXCR4, dominant negative CCL2, EGF, exosomes, FGF-2, GATA-4 GDF-11, GDNF, hCG, HGF, HIF-1alpha, HLA-G, HO-1, hTERT, IFN-b, IFT-1, LIGHT, miR-126, NK4, NUR77, OCT-4, PGE-1, SDF-1, STC-1, TERT, TRAIL, VEGF, WNT11, XIAP, and a combination thereof.

Embodiments of the disclosure include kits that comprising part or all of any system encompassed herein.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

For a more complete understanding of the present disclosure, reference is now made to the following descriptions taken in conjunction with the accompanying drawings.

FIG. 1 is one example of a system of the disclosure having a mammal as the subject.

FIG. 2 is one example of a system of the disclosure having an organ or tissue as the subject.

FIG. 3 shows assessment of aging where foreskin fibroblasts were exposed to an accelerated senescence protocol induced by exposure to indicated concentrations of H₂O₂ for 48 hours. Cells were cultured in control media (RPMI) (left bar of groupings of three bars) or 5% conditioned media (middle bar of groupings of three bars), or 10% conditioned media (right bar of groupings of three bars).

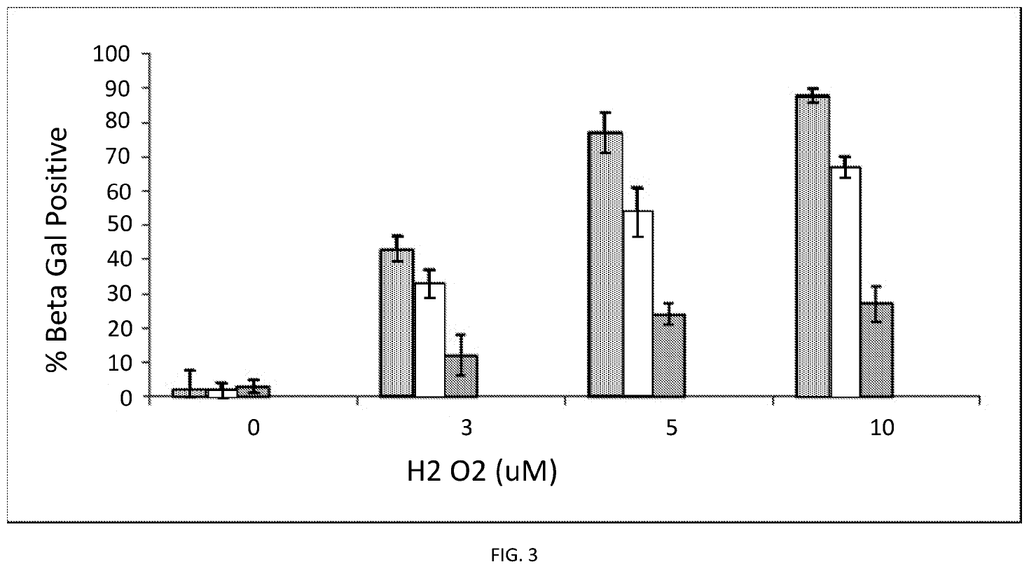

### DETAILED DESCRIPTION

### I. EXAMPLES OF DEFINITIONS

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein.

As used herein, the term “about” or “approximately” refers to a quantity, level, value, number, frequency, percentage, dimension, size, amount, weight or length that varies by as much as 30, 25, 20, 25, 10, 9, 8, 7, 6, 5, 4, 3, 2 or 1% to a reference quantity, level, value, number, frequency, percentage, dimension, size, amount, weight or length. In particular embodiments, the terms “about” or “approximately” when preceding a numerical value indicates the value plus or minus a range of 15%, 10%, 5%, or 1%. With respect to biological systems or processes, the term can mean within an order of magnitude, preferably within 5-fold, and more preferably within 2-fold, of a value. Unless otherwise stated, the term ‘about’ means within an acceptable error range for the particular value.

As used herein, the term “aging” refers to a subject that has begun to display signs of age-associated degeneration. Examples of age-associated degeneration include decreased telomere length, enhanced oxidative stress, formation of cellular tangles, generation of taupathy in the central nervous system, and/or increased number of proteins that are misfolding.

As used herein, the term “bioreactor” refers to a vessel or container that houses or is capable of housing cells, cellular components, and/or other biological material, for example. The bioreactor can be comprised of any suitable material. In some embodiments, the bioreactor is comprised, at least in part, of at least one semi-permeable membrane described herein. The bioreactor of the disclosure may comprise an environment permitting cellular viability while allowing for tissue culture media to perfuse viable cells in a manner allowing for tissue culture media to remove growth factors and therapeutic agents from the cells growing in the environment. In one embodiment, the cells are perfused with plasma from the blood of an individual in need of treatment.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

As used herein, the term “degenerative” or “degenerative factors” refers to one or agents that are detrimental to a subject. Such agent(s) could be one or more age-associated factors including at least one or more inflammation molecules, for example inflammatory molecules selected from the group consisting of IL-1, TNF-alpha, IL-6, IL-17, IL33, and a combination thereof, or other factors known in the art.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the term “factors” refers to molecules, such as proteins, lipids, nucleic acids, metabolites, hormones, biosynthetic products, or other molecules produced by a subject and/or bioreactor.

The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

As used herein, the term “regenerative” or “regenerative factors” refers to one or more agents that act to restore, either fully or partially, a subject to the state prior to being subject to degenerative stimuli. Such regenerative stimuli can be said to act inversely to associated degenerative agent(s), for example.

As used herein, the term “selectively permeable membrane” refers to one or more devices or materials that can selectively permit the passage of factors while restricting the passage of cells. In some embodiments, the selectively permeable membrane utilized is a hollow fiber membrane. Hollow fiber membranes are semi-permeable membranes comprised of long, porous filaments with an inside and an outside that can selectively allow the passage of specific compositions from the inside to the outside or from the outside to the inside while restricting such passage for other compositions. In some embodiments, a hollow fiber membrane allows for the passage of factors while restricting the passage of cells. In particular embodiments, the membrane accomplishes two things:

In particular embodiments, the membrane does not allow blood cells from the individual to come into contact with the cells in the bioreactor: only the plasma from the blood of the individual contacts the regenerative cells in the bioreactor. In specific cases, the membrane does not allow the cells from the bioreactor to leak into the blood of the individual.

The term “cellular senescence” (or merely “senescence”) is a stress-induced, durable cell cycle arrest of previously replication-competent cells.

“Treatment,” “treat,” or “treating” means a method of reducing the effects of a disease or condition. Treatment can also refer to a method of reducing the disease or condition itself rather than just the symptoms. The treatment can be any reduction from pre-treatment levels and can be but is not limited to the complete ablation of the disease, condition, or the symptoms of the disease or condition. Therefore, in the disclosed methods, treatment” can refer to a 10%, 20%, 30%, 40%, 50%, 60%, 70%, 80%, 90%, or 100% reduction in the severity of an established disease or the disease progression, including reduction in the severity of at least one symptom of the disease. For example, a disclosed method for reducing the immunogenicity of cells is considered to be a treatment if there is a detectable reduction in the immunogenicity of cells when compared to pre-treatment levels in the same subject or control subjects. Thus, the reduction can be a 10, 20, 30, 40, 50, 60, 70, 80, 90, 100%, or any amount of reduction in between as compared to native or control levels. It is understood and herein contemplated that “treatment” does not necessarily refer to a cure of the disease or condition, but an improvement in the outlook of a disease or condition. In specific embodiments, treatment refers to the lessening in severity or extent of at least one symptom and may alternatively or in addition refer to a delay in the onset of at least one symptom.

In some embodiments of the disclosure, the cells generated and/or utilized are from a “young subject”, which may be a subject less than 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 year of age.

In specific embodiments, a subject that lacks one or more signs of aging may be of no particular age but may be less than 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 year of age.

### II. EXTRACORPOREAL SYSTEM

In particular embodiments, the disclosure describes a system possessing the ability to induce one or more regenerative changes in a subject, such as systemically or locally in a subject. In some embodiments, the system generates regenerative feedback proportional to a degree of degenerative stimuli, and in particular aspects can cancel one or more degenerative stimuli effects. In particular embodiments, the device produces, releases, or secretes one or more unit(s) of regenerative factor(s) for every one or more unit(s) of degenerative factor(s) detected by the system. In some cases, the degenerative stimuli comprises aging-associated stimuli and/or aging-associated inflammation. More precisely, one or more than one of several inflammatory mediators are associated with various aspects of aging or degeneration. Without being bound by theory, particular embodiments of systems encompassed herein represent an exogenous bioreactor in which regenerative cells in the system are in contact with circulating factors from a subject linked to the bioreactor, and the cells in the system produce one or more regenerative factors in response to the circulating factors in circulation in the system. In some embodiments of the disclosure, circulating factors from a subject are age-associated factors, such as inflammatory mediators. It is the object of the invention to create an artificial environment where inflammatory or other degenerative can trigger generation of regenerative factors based on the need of the body. In some embodiments of the disclosure, regenerative factors include agents such as GDF-11, exosomes, or other agents associated with regeneration, such as BDNF, EGF, hCG, VEGF, and IGF-1.

In some embodiments, the subject may be aging. The aging may be natural aging or may be accelerated aging, or aging may be the time dependent decline of physiological function. In some embodiments, aging is associated with enhanced fibrosis, enhanced inflammation, and/or reduced telomere length, all compared to normal. In some embodiments, the subject may have abnormal levels of degenerative factors associated with a disease state. In some embodiments the subject may have had a certain trauma or injury or medical condition that changed the levels of degenerative and/or regenerative factors in the subject.

In certain embodiments, the subject may be an organ from an individual that is aging, or has a disease, or has had a trauma or injury, for example. In other embodiments, the subject may be an organ that will be used for transplantation. In cases of transplantation, the system is used to keep the organ viable. In further embodiments the subject may be a tissue or set of tissues to be kept viable. In embodiments of the disclosure, wherein the subject is an organ, the subject may be any organ including liver, pancreas, gallbladder, stomach, small intestine, large intestine, lung, kidney, heart, spleen, brain, eye, omentum, subintestinal mucusa, or any other organ, or any part of an organ, or any part of an organ system.

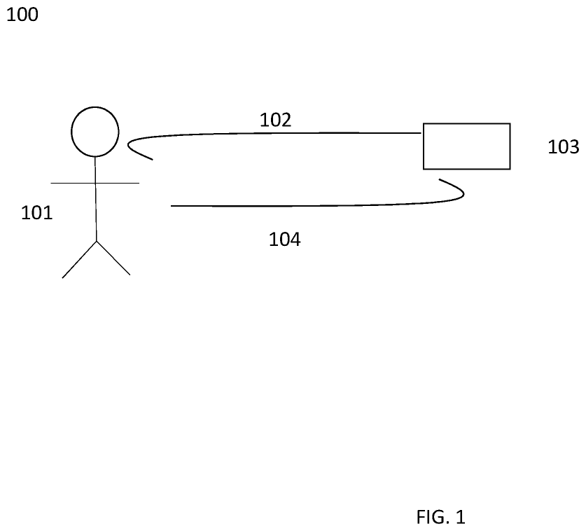

Turning to FIG. 1, system 100 illustrates one example of an extracorporeal system for preventing or reversing aging in subject 101 and/or treating a medical condition in subject 101. In one example of the system, the circulatory system of subject 101 is attached in-line to tubing 102 such that the tubing 102 is capable of transferring blood or plasma from subject 101 extracorporeally. Tubing 102 transfers the blood or plasma from subject 101 to bioreactor 103 that houses cells capable of detecting the presence of one or more degenerative factors in the blood or plasma from subject 101. Upon exposure of the cells in bioreactor 103 to the blood or plasma from subject 101 transferred through tubing 102, the cells in bioreactor 103 produce one or more regenerative factor(s). The one or more regenerative factor(s) from the cells in bioreactor 103 then are transferred within the blood or plasma through tubing 104 back to the subject 101, because tubing 104 is in-line with the system and is attached to the circulatory system of subject 101. A semi-permeable membrane may be incorporated at any location in the system, such as in or throughout tubing 102 and/or 104 or may comprise, at least in part, bioreactor 103.

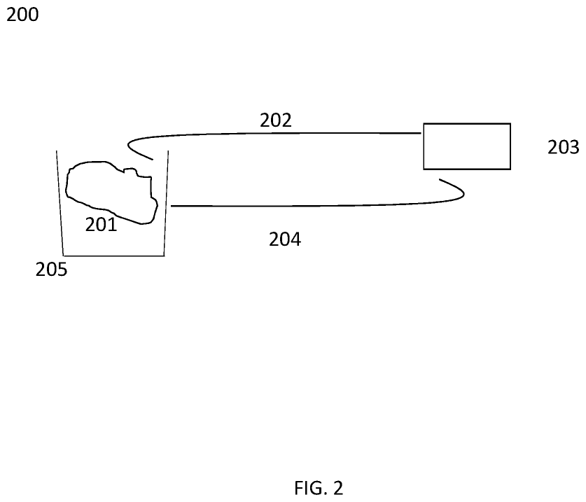

Turning to FIG. 2, system 200 illustrates one example of an extracorporeal system for preventing or reversing aging in organ or tissue 202 and/or treating a medical condition in organ or tissue 201. In one example of the system, the circulatory system of organ or tissue 201 is attached in-line to tubing 202 such that the tubing 202 is capable of transferring blood or plasma from organ or tissue 201 outside of the respective organ or tissue. The organ or tissue may be contained in container 205. Tubing 202 transfers organ preservation solution (which is commercially available) from organ or tissue 201 to bioreactor 203 that houses cells capable of detecting the presence of one or more degenerative factors in the blood or plasma from organ or tissue 201. Upon exposure of the cells in bioreactor 203 to the blood or plasma from organ or tissue 201 transferred through tubing 202, the cells in bioreactor 203 produce one or more regenerative factor(s). The one or more regenerative factor(s) from the cells in bioreactor 203 then are transferred within the blood or plasma through tubing 204 back to the organ or tissue 201, because tubing 204 is in-line with the system and is attached to the circulatory system of organ or tissue 201. A semi-permeable membrane may be incorporated at any location in the system, such as in or throughout tubing 202 and/or 204 or may comprise, at least in part, bioreactor 203 and/or container 205.

In particular embodiments of the disclosure, a bioreactor is provided comprising a compartment suitable for housing regenerative biological material, including cells. In some embodiments, the bioreactor comprises at least one selectively permeable membrane that may be in contact with the cells. In particular embodiments, the bioreactor has at least one selectively permeable wall. In particular embodiments, the selectively permeable membrane can be a selectively-permeable hollow fiber. The bioreactor may comprise a plurality of selectively permeable hollow fibers passing through the compartment through which one or both of a gas and a fluid comprising nutrients for the cells can be passed. In certain embodiments, the bioreactor comprises a plurality of selectively permeable hollow fibers passing through the bioreactor. In other embodiments, the selectively permeable membrane or plurality of selectively permeable membranes are separate from the bioreactor. In specific embodiments, the membrane(s) are placed in the tubing connecting at least one of the bioreactors and the subject. The bioreactor and selectively permeable membrane may be connected to the subject by tubing. The tubing may be flexible or rigid and made of any material that is biocompatible. In some embodiments, the tubing is connected to the circulatory system of the subject at least at one point of the circulatory system of the subject. In particular embodiments, the tubing is connected to a container that houses the subject (for example, when the subject is a tissue or organ). In such embodiments, the tubing allows for circulation of fluid from the subject and/or allows for circulation of fluid around the subject. In particular embodiments, a method and/or device to circulate the fluid in the tubing connecting the subject, bioreactor, and selectively permeable membrane is used.

The biological material housed in the bioreactor may serve to 1) sense the levels of a given degenerative factor(s); 2) produce the appropriate levels of one or more appropriate counteracting regenerative factor(s); and 3) in some embodiments, also release diagnostic markers that would serve to delineate the degree of degenerative factor(s) produced by the subject. Biological material for use with the device may be any cell or cellular material or plurality of cells that is effective in its use in the bioreactor, and may be xenogeneic, syngeneic, allogeneic, or autologous cells with respect to an individual treated by use of the bioreactor. In particular embodiments, wherein the biological material are cells, the cells may detect degenerative factors and secrete regenerative factors at a rate commensurate with the rate of degenerative factor detection. The commensurate rate may be a ratio of regenerative secretion to degenerative detection. The ratio may be 50:1, 10:1, 5:1, 1:1, 1:5, 1:10, 1:50, and any ratio between, for example.

In specific embodiments, the biological material comprises cells. In certain embodiments, the cells are cultured in the bioreactor. In other embodiments, the cells are cultured outside of the bioreactor, in a suitable cell culture container, then harvested for use in the bioreactor. In certain embodiments, the cells utilized are fibroblasts. The cells used, including when they may be fibroblasts, may have one or more surface markers selected from the group consisting of CD73, CD90, CD56, SSEA3, SSEA4, Tra-1-60, Tra-1-81, Tra-2-54, HLA class I, CD13, CD44, CD49b, CD105, aminopeptidase N, hyaluronic acid-binding receptor, collagen/laminin-binding integrin alpha 2, and a combination thereof. At least about 80%, 85%, 90%, 95%, 96%, 97%, 98%, 99%, or about 100% of the population of cells used in the disclosure may express one or more specific surface markers. The cells used may also lack one or more surface markers, for example one or more selected from the group consisting of CD14, CD34, CD45, HLA Class II, and a combination thereof. In particular embodiments of the disclosure, the cells are capable of self-renewal in tissue culture, maintain euploidy for >1 year in culture, share markers with human ES cells, and/or are capable of differentiating into all three germ layers of the developing embryo. In a particular embodiment, the cells are fibroblast cells that are obtainable in the amnion harvested during the second trimester of human pregnancies. It is known that fibroblasts comprise multiple morphologically-distinguishable cell types, the majority of the cells are prone to senescence and are lost from cultures. In one embodiment, fibronectin coated plates and culture conditions such as are described in U.S. Pat. No. 7,569,385 (incorporated by reference herein) are used to grow cells from fibroblast harvests from normal 16-18 week pregnancies. In one embodiment, the cells of the disclosure are of fetal origin and may have a normal diploid karyotype. The regenerative cells may be isolated from any mammal, including any primate, including humans. However, the fibroblast regenerative cells may be isolated in a similar manner from other species. Examples of species that may be used to derive the fibroblast regenerative cells include but are not limited to mammals, humans, primates, dogs, cats, goats, elephants, sheep, endangered species, cattle, horses, pigs, mice, rabbits, and the like.

The cells used in the disclosure can be recognized by specific cell surface proteins or by the presence and/or lack of specific cellular proteins. Typically, specific cell types have specific cell surface proteins. These surface proteins can be used as markers to determine or confirm specific cell types. Typically, these surface markers can be visualized using antibody-based technology or other detection methods. In specific embodiments, the markers are selected from the group consisting of CD73, CD90, CD56, SSEA3, SSEA4, Tra-1-60, Tra-1-81, Tra-2-54, HLA class I, CD13, CD44, CD49b, CD105, aminopeptidase N, hyaluronic acid-binding receptor, collagen/laminin-binding integrin alpha 2, and a combination thereof.

In one particular embodiment of the disclosure, the cells are human stem cells that can be propagated for an indefinite period of time in continuous culture in an undifferentiated state. The term “undifferentiated” refers to cells that have not become specialized cell types. The cells are cultured in a nutrient medium. The nutrient medium may comprise any one or more of the following in an appropriate combination: isotonic saline, buffer, amino acids, antibiotics, serum or serum replacement, and exogenously added factors. The cells may be grown in an undifferentiated state for as long as desired, and can then be cultured under certain conditions to allow progression to a differentiated or activated state. In some embodiments the cells are cultured in an undifferentiated state for at least 1, 2, 3, 4, 5, 6, 7, 8, 9, 10, 11, 12, 13, 14, 15, 16, 17, 18, 19, 20, 21, 22, 23, 24, 25 or more days before activating the cells. In specific embodiments, the cells are cultured in the presence of factor(s) before activating the cells. In further embodiments the cells are cultured in the presence of factor(s) that may include nerve growth factor, bFGF, dibutryl cAMP, IBMX, retinoic acid, exendin-4, or other factors either alone or in combination that are useful for producing the desired activated cells. By activated, the term is meant the process whereby an unspecialized cell acquires the features of a specialized cell such as a heart, liver, muscle, pancreas or other organ or tissue cell. The cells in the present disclosure may be activated into any useable cell type for the described system.

General methods relating to cell differentiation techniques that may be useful for differentiating the cells of this disclosure can be found in general texts such as: Teratocarcinomas and embryonic stem cells: A practical approach (E. J. Robertson, ed., IRL Press Ltd. 1987); Guide to Techniques in Mouse Development (P. M. Wasserman et al. eds., Academic Press 1993); Embryonic Stem Cell Differentiation in vitro (M. V. Wiles, Meth. Enzymol. 225:900, 1993); Properties and uses of Embryonic Stem Cells: Prospects for Application to Human Biology and Gene Therapy (P. D. Rathjen et al., Reprod. Fertil. Dev. 10:31, 1998); and in Stem cell biology (L. M. Reid, Curr. Opinion Cell Biol. 2:121, 1990), each of which is incorporated by reference herein in its entirety.

In some embodiments of the disclosure, the cells are cultured in the presence of one or more cytokines in order to activate the cell for use in the system. In particular embodiments, the cytokine is either IL-1, or IFNgamma, or both concurrently or sequentially. In some embodiments, the concentration of IL-1 supplied to the cell culture may be 1-100 ng/mL, 5-100 ng/mL, 10-100 ng/mL, or 20-40 ng/mL, for example. In other embodiments, the concentration of IL-1 may be 1 ng/mL, 5 ng/mL, 10 ng/mL, 20 ng/mL, 30 ng/mL, 40 ng/mL, 50 ng/mL, 60 ng/mL, 70 ng/mL, 80 ng/mL, 90 ng/mL, or 100 ng/mL, for example. In some embodiments, the concentration of IFNgamma may be 1-1000 IU, 5-1000 IU, 10-1000 IU, 1-500 IU, 5-500 IU, 10-500 IU, 100-500 IU, or 250 IU, for example. In further embodiments, the concentration IFNgamma may be 1 IU, 5 IU, 10 IU, 50 IU, 100 IU, 200 IU, 250 IU, 300 IU, 400 IU, 500 IU, 600 IU, 700 IU, 800 IU, 900 IU, or 1000 IU, for example.

In particular embodiments, cells may be cultured prior to use in the system. In specific cases, the culturing of cells induces the expression of complement inhibitory molecules. These molecules may include CD35, CD46, C4BP, CD55, Factor H, or other factors that reduce the activation of the complement system.

In some embodiments of the disclosure, the cells are transfected to possess enhanced regenerative properties. The transfection(s) may be accomplished by use of one or more viral vectors (for example, retroviral, lentiviral, adenoviral, adeno-associated viral) or one or more non-viral vectors (for example, plasmids). Means to perform transfections are well-known in the art and discussed in the following references [3-9]. Specific examples of genes include: SDF-1 to promote stem cell homing, particularly hematopoietic stem cells [10], GDNF to treat Parkinson's in an animal model [11], HGF to accelerate remyelination in a brain injury model [12], akt to protect against pathological cardiac remodeling and cardiomyocyte death [13], TRAIL to induce apoptosis of tumor cells [14-17], PGE-1 synthase for cardioprotection [18], NUR77 to enhance migration [19], BDNF to reduce ocular nerve damage in response to hypertension [20], HIF-1 alpha to stimulate osteogenesis [21], dominant negative CCL2 to reduce lung fibrosis [22], interferon beta to reduce tumor progression [23], HLA-G to enhance immune suppressive activity [24], hTERT to induce differentiation along the hepatocyte lineage [25], cytosine deaminase [26], OCT-4 to reduce senescence [27, 28], BAMBI to reduce TGF expression and protumor effects [29], HO-1 for radioprotection [30], LIGHT to induce antitumor activity [31], miR-126 to enhance angiogenesis [32, 33], bcl-2 to induce generation of nucleus pulposus cells [34], telomerase to induce neurogenesis [35], CXCR4 to accelerate hematopoietic recovery [36] and reduce unwanted immunity [37], wnt11 to promote regenerative cytokine production [38], and the HGF antagonist NK4 to reduce cancer [39]. Other factors known in the art to reduce degeneration or induce regeneration may also be used.

### III. METHODS OF TREATING A MEDICAL CONDITION OR REVERSING OR SLOWING AGING

Embodiments of the disclosure include methods of treating a medical condition or reducing the effects of the natural process of aging. The medical condition may be a senescence-related disorder or age-related disorder or process. An individual may have cellular accumulation of damage and other deleterious changes, including loss of at least some cells and structures, such as compared to a younger individual. In specific cases, the individual has lost the ability for adult somatic cells to be converted to partially reprogrammed cells or induced pluripotent stem cells for the individual.

In some embodiments of the disclosure, the methods and systems encompass extension of cellular and/or organismal lifespan, compared to an individual that has not been subjected to methods and systems of the disclosure. In specific embodiments, the methods and systems of the disclosure allow for reversal or slowing of cellular senescence, the irreversible loss of replicative capacity in somatic cells. In specific cases, the methods and systems prevent or reduce telomere dysfunction and/or alterations in mitochondrial homeostasis associated with cellular senescence.

In particular embodiments, the medical condition is an aging-associated disease, such as a disease that is most often seen with increasing frequency with increasing senescence. In at least some cases, aging-associated diseases are complications stemming from senescence. In specific embodiments, age-associated diseases may be distinguished from the aging process itself, because all adult animals age, save for a few rare exceptions, but not all adult animals experience all age-associated diseases. In specific cases, aging-associated diseases include neurodegenerative diseases, atherosclerosis, cardiovascular disease, cancer, arthritis, cataracts, osteoporosis, type 2 diabetes, hypertension, Parkinson's Disease, amyotrophic lateral sclerosis, and/or Alzheimer's disease.

### IV. EXAMPLES

The following examples are presented in order to more fully illustrate the preferred embodiments of the disclosure. They should in no way, however, be construed as limiting the broad scope of the disclosure.

### Example 1

### Harvesting Cells for Use in the System

The surface markers of the isolated multipotent amniotic fluid stem cells (MAFSC) cells derived from independently-harvested fibroblast samples were tested for a range of cell surface and other markers, using monoclonal antibodies and FACS analysis. These cells can be characterized by the following cell surface markers: SSEA3, SSEA4, Tra-1-60, Tra-1-81, Tra-2-54. The MAFSC cells can be distinguished from mouse ES cells in that the MAFSC cells do not express the cell surface marker SSEA1. Additionally, MAFSC express the stem cell transcription factor Oct-4. The MAFSC cells can be recognized by the presence of at least one, or at least two, or at least three, or at least four, or at least five, or at least six, or all of the following cellular markers SSEA3, SSEA4, Tra-1-60, Tra-1-81, Tra-2-54 and Oct-4.

The MAFSC cultures express very little or no SSEA-1 marker. In addition to the embryo stem cell markers SSEA3, SSEA4, Tra1-60, Tra1-81, Tra2-54, Oct-4 the fibroblast regenerative cells also expressed high levels of the cell surface antigens that are normally found on human mesenchymal stem cells, but not normally on human embryo stem cells. This set of markers includes CD13 (99.6%) aminopeptidase N, CD44 (99.7%) hyaluronic acid-binding receptor, CD49b (99.8%) collagen/laminin-binding integrin alpha2, and CD105 (97%) endoglin. The presence of both the embryonic stem cell markers and the hMSC markers on the MAFSC cell cultures indicates that fibroblast-derived MAFSC cells, grown and propagated as described here, represent a novel class of human stem cells that combined the characteristics of hES cells and of hMSC cells.

In order to determine the quality of MAFSC cultures, flow cytometry is performed on all cultures for surface expression of SH-2, SH-3, SH-4 MSC markers and lack of contaminating CD14− and CD-45 positive cells. Cells were detached with 0.05% trypsin-EDTA, washed with DPBS+2% bovine albumin, fixed in 1% paraformaldehyde, blocked in 10% serum, incubated separately with primary SH-2, SH-3 and SH-4 antibodies followed by PE-conjugated anti-mouse IgG(H+L) antibody. Confluent MSC in 175 cm² flasks are washed with Tyrode's salt solution, incubated with medium 199 (M199) for 60 min, and detached with 0.05% trypsin-EDTA (Gibco). Cells from 10 flasks were detached at a time and MSCs were resuspended in 40 ml of M199+1% human serum albumin (HSA; American Red Cross, Washington D.C., USA). MSCs harvested from each 10-flask set were stored for up to 4 h at 4° C. and combined at the end of the harvest. A total of 2-10′10⁶ MSC/kg were resuspended in M199+1% HSA and centrifuged at 460 g for 10 min at 20° C. Cell pellets were resuspended in fresh M199+1% HSA media and centrifuged at 460 g for 10 min at 20° C. for three additional times. Total harvest time was 2-4 h based on MSC yield per flask and the target dose. Harvested MSC were cryopreserved in Cryocyte (Baxter, Deerfield, Ill., USA) freezing bags using a rate controlled freezer at a final concentration of 10% DMSO (Research Industries, Salt Lake City, Utah, USA) and 5% HSA.

In one embodiment of the disclosure, the MAFSC are allowed to adhere for 72 h followed by media changes every 3-4 days. Adherent cells are removed with 0.05% trypsin-EDTA and replated at a density of 1′10⁶ per 175 cm². The MAFSC may be administered intravenously, or in a preferred embodiment, intrathecally in a patient suffering radiation associated neurodegenerative manifestations. Although doses may be determined by one of skill in the art, and are dependent on various patient characteristics, intravenous administration may be performed at concentrations ranging from 1-10 million MSC per kilogram, with a preferred dose of approximately 2-5 million cells per kilogram.

### Example 2

### Examples of Transfected Cells for the System

In one embodiment of the invention MAFSC are transfected with anti-apoptotic proteins to enhance in vivo longevity. The present disclosure includes a method of using MAFSC that have been cultured under conditions to express increased amounts of at least one anti-apoptotic protein as a therapy to inhibit or prevent apoptosis. In one embodiment, the MAFSC which are used as a therapy to inhibit or prevent apoptosis have been contacted with an apoptotic cell. The invention is based on the discovery that MAFSC that have been contacted with an apoptotic cell express high levels of anti-apoptotic molecules. In some instances, the MAFSC that have been contacted with an apoptotic cell secrete high levels of at least one anti-apoptotic protein, including but not limited to, STC-1, BCL-2, XIAP, Survivin, and Bcl-2XL. Methods of transfecting antiapoptotic genes into MAFSC have been previously described which can be applied to the current invention, said antiapoptotic genes that can be utilized for practice of the invention, in a non-limiting way, include GATA-4 [40], FGF-2 [41], bcl-2 [34, 42], and HO-1 [43]. Based upon the disclosure provided herein, MAFSC can be obtained from any source. The MAFSC may be autologous with respect to the subject and/or a recipient (obtained from the same host) or allogeneic with respect to the subject and/or a recipient. In addition, the MAFSC may be xenogeneic to the subject and/or a recipient (obtained from an animal of a different species). In one embodiment of the invention MAFSC are pretreated with agents to induce expression of antiapoptotic genes, one example is pretreatment with exendin-4 as previously described [44]. In a further non-limiting embodiment, MAFSC used in the present invention can be isolated, from the bone marrow of any species of mammal, including but not limited to, human, mouse, rat, ape, gibbon, bovine. In a non-limiting embodiment, the MAFSC are isolated from a human, a mouse, or a rat. In another non-limiting embodiment, the MAFSC are isolated from a human.

### Example 3

### Fibroblast Conditioned Media Reduces Senescence

Foreskin fibroblasts were obtained from American Type Culture Collection (ATCC) and cultured according to manufacturer instructions. To generate fibroblast conditioned media, fibroblasts were isolated based on expression of CD73 and cultured at a concentration of 1 million cells per 10 ml of RPMI media with 10% fetal calf serum for 24 hours and media was used as conditioned media.

To assess aging foreskin fibroblasts were exposed to an accelerated senescence protocol induced by exposure to indicated concentrations of H₂O₂ for 48 hours. Cells were cultured in control media (RPMI) (left bar of groupings of three bars) or 5% conditioned media (middle bar of groupings of three bars), or 10% conditioned media (right bar of groupings of three bars). Senescence was detected by fixing cells with 4% paraformaldehyde and SA-β-Gal was stained using senescent cells histochemical staining kit (Sigma Aldrich, St. Louis, Mo., USA). Three images per each well were collected, and the SA-β-Gal-stained cells were counted.

Reduction of senescence associated beta galactosidase was observed with conditioned media from CD73-selected fibroblasts.

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

### REFERENCES

All patents and publications mentioned in this specification are indicative of the level of those skilled in the art to which the invention pertains. All patents and publications herein are incorporated by reference to the same extent as if each individual publication was specifically and individually indicated to be incorporated by reference in their entirety.

1. Conboy, I. M., et al., Rejuvenation of aged progenitor cells by exposure to a young systemic environment. Nature, 2005. 433(7027): p. 760-4.

2. Katsimpardi, L., et al., Vascular and neurogenic rejuvenation of the aging mouse brain by young systemic factors. Science, 2014. 344(6184): p. 630-4.

3. Zhang, X. Y., et al., Lentiviral vectors for sustained transgene expression in human bone marrow-derived stromal cells. Mol Ther, 2002. 5(5 Pt 1): p. 555-65.

4. Kyriakou, C. A., et al., Human mesenchymal stem cells (hMSCs) expressing truncated soluble vascular endothelial growth factor receptor (tsFlk-1) following lentiviral-mediated gene transfer inhibit growth of Burkitt's lymphoma in a murine model. J Gene Med, 2006. 8(3): p. 253-64.

5. Worsham, D. N., et al., In vivo gene transfer into adult stem cells in unconditioned mice by in situ delivery of a lentiviral vector. Mol Ther, 2006. 14(4): p. 514-24.

6. Rabin, N., et al., A new xenograft model of myeloma bone disease demonstrating the efficacy of human mesenchymal stem cells expressing osteoprotegerin by lentiviral gene transfer. Leukemia, 2007. 21(10): p. 2181-91.

7. Kallifatidis, G., et al., Improved lentiviral transduction of human mesenchymal stem cells for therapeutic intervention in pancreatic cancer. Cancer Gene Ther, 2008. 15(4): p. 231-40.

8. Meyerrose, T. E., et al., Lentiviral-transduced human mesenchymal stem cells persistently express therapeutic levels of enzyme in a xenotransplantation model of human disease. Stem Cells, 2008. 26(7): p. 1713-22.

9. McGinley, L., et al., Lentiviral vector mediated modification of mesenchymal stem cells & enhanced survival in an in vitro model of ischaemia. Stem Cell Res Ther, 2011. 2(2): p. 12.

10. Liang, X., et al., Human bone marrow mesenchymal stem cells expressing SDF-1 promote hematopoietic stem cell function of human mobilised peripheral blood CD34+ cells in vivo and in vitro. Int J Radiat Biol, 2010. 86(3): p. 230-7.

11. Glavaski-Joksimovic, A., et al., Glial cell line-derived neurotrophic factor-secreting genetically modified human bone marrow-derived mesenchymal stem cells promote recovery in a rat model of Parkinson's disease. J Neurosci Res, 2010. 88(12): p. 2669-81.

12. Liu, A. M., et al., Umbilical cord-derived mesenchymal stem cells with forced expression of hepatocyte growth factor enhance remyelination and functional recovery in a rat intracerebral hemorrhage model. Neurosurgery, 2010. 67(2): p. 357-65; discussion 365-6.

13. Yu, Y. S., et al., AKT-modified autologous intracoronary mesenchymal stem cells prevent remodeling and repair in swine infarcted myocardium. Chin Med J (Engl), 2010. 123(13): p. 1702-8.

14. Mueller, L. P., et al., TRAIL-transduced multipotent mesenchymal stromal cells (TRAIL-MSC) overcome TRAIL resistance in selected CRC cell lines in vitro and in vivo. Cancer Gene Ther, 2011. 18(4): p. 229-39.

15. Yan, C., et al., Suppression of orthotopically implanted hepatocarcinoma in mice by umbilical cord-derived mesenchymal stem cells with sTRAIL gene expression driven by AFP promoter. Biomaterials, 2014. 35(9): p. 3035-43.

16. Deng, Q., et al., TRAIL-secreting mesenchymal stem cells promote apoptosis in heat-shock-treated liver cancer cells and inhibit tumor growth in nude mice. Gene Ther, 2014. 21(3): p. 317-27.

17. Sage, E. K., et al., Systemic but not topical TRAIL-expressing mesenchymal stem cells reduce tumour growth in malignant mesothelioma. Thorax, 2014. 69(7): p. 638-47.

18. Lian, W. S., et al., In vivo therapy of myocardial infarction with mesenchymal stem cells modified with prostaglandin I synthase gene improves cardiac performance in mice. Life Sci, 2011. 88(9-10): p. 455-64.

19. Maijenburg, M. W., et al., Nuclear receptors Nur77 and Nurr1 modulate mesenchymal stromal cell migration. Stem Cells Dev, 2012. 21(2): p. 228-38.

20. Harper, M. M., et al., Transplantation of BDNF-secreting mesenchymal stem cells provides neuroprotection in chronically hypertensive rat eyes. Invest Ophthalmol Vis Sci, 2011. 52(7): p. 4506-15.

21. Zou, D., et al., In vitro study of enhanced osteogenesis induced by HIF-1alpha-transduced bone marrow stem cells. Cell Prolif, 2011. 44(3): p. 234-43.

22. Saito, S., et al., Mesenchymal stem cells stably transduced with a dominant-negative inhibitor of CCL2 greatly attenuate bleomycin-induced lung damage. Am J Pathol, 2011. 179(3): p. 1088-94.

23. Seo, K. W., et al., Anti-tumor effects of canine adipose tissue-derived mesenchymal stromal cell-based interferon-beta gene therapy and cisplatin in a mouse melanoma model. Cytotherapy, 2011. 13(8): p. 944-55.

24. Yang, H. M., et al., Enhancement of the immunosuppressive effect of human adipose tissue-derived mesenchymal stromal cells through HLA-G1 expression. Cytotherapy, 2012. 14(1): p. 70-9.

25. Liang, X. J., et al., Differentiation of human umbilical cord mesenchymal stem cells into hepatocyte-like cells by hTERT gene transfection in vitro. Cell Biol Int, 2012. 36(2): p. 215-21.

26. Fei, S., et al., The antitumor effect of mesenchymal stem cells transduced with a lentiviral vector expressing cytosine deaminase in a rat glioma model. J Cancer Res Clin Oncol, 2012. 138(2): p. 347-57.

27. Jaganathan, B. G. and D. Bonnet, Human mesenchymal stromal cells senesce with exogenous OCT4. Cytotherapy, 2012. 14(9): p. 1054-63.

28. Han, S. H., et al., Effect of ectopic OCT4 expression on canine adipose tissue-derived mesenchymal stem cell proliferation. Cell Biol Int, 2014. 38(10): p. 1163-73.

29. Shangguan, L., et al., Inhibition of TGF-beta/Smad signaling by BAMBI blocks differentiation of human mesenchymal stem cells to carcinoma-associated fibroblasts and abolishes their protumor effects. Stem Cells, 2012. 30(12): p. 2810-9.

30. Kearns-Jonker, M., et al., Genetically Engineered Mesenchymal Stem Cells Influence Gene Expression in Donor Cardiomyocytes and the Recipient Heart. J Stem Cell Res Ther, 2012. S1.

31. Ma, G. L., et al., [Study of inhibiting and killing effects of transgenic LIGHT human umbilical cord blood mesenchymal stem cells on stomach cancer]. Zhonghua Wei Chang Wal Ke Za Zhi, 2012. 15(11): p. 1178-81.

32. Huang, F., et al., Mesenchymal stem cells modified with miR-126 release angiogenic factors and activate Notch ligand Delta-like-4, enhancing ischemic angiogenesis and cell survival. Int J Mol Med, 2013. 31(2): p. 484-92.

33. Huang, F., et al., Overexpression of miR-126 promotes the differentiation of mesenchymal stem cells toward endothelial cells via activation of PI3K/Akt and MAPK/ERK pathways and release of paracrine factors. Biol Chem, 2013. 394(9): p. 1223-33.

34. Fang, Z., et al., Differentiation of GFP-Bcl-2-engineered mesenchymal stem cells towards a nucleus pulposus-like phenotype under hypoxia in vitro. Biochem Biophys Res Commun, 2013. 432(3): p. 444-50.

35. Madonna, R., et al., Transplantation of mesenchymal cells rejuvenated by the overexpression of telomerase and myocardin promotes revascularization and tissue repair in a murine model of hindlimb ischemia. Circ Res, 2013. 113(7): p. 902-14.

36. Zang, Y., et al., [Influence of CXCR4 overexpressed mesenchymal stem cells on hematopoietic recovery of irradiated mice]. Zhongguo Shi Yan Xue Ye Xue Za Zhi, 2013. 21(5): p. 1261-5.

37. Cao, Z., et al., Protective effects of mesenchymal stem cells with CXCR4 up-regulation in a rat renal transplantation model. PLoS One, 2013. 8(12): p. e82949.

38. Liu, S., et al., Overexpression of Wnt11 promotes chondrogenic differentiation of bone marrow-derived mesenchymal stem cells in synergism with TGF-beta. Mol Cell Biochem, 2014. 390(1-2): p. 123-31.

39. Zhu, Y., et al., Mesenchymal stem cell-based NK4 gene therapy in nude mice bearing gastric cancer xenografts. Drug Des Devel Ther, 2014. 8: p. 2449-62.

40. Yu, B., et al., Enhanced mesenchymal stem cell survival induced by GATA-4 overexpression is partially mediated by regulation of the miR-15 family. Int J Biochem Cell Biol, 2013. 45(12): p. 2724-35.

41. Xu, W., et al., Basic fibroblast growth factor expression is implicated in mesenchymal stem cells response to light-induced retinal injury. Cell Mol Neurobiol, 2013. 33(8): p. 1171-9.

42. Li, W., et al., Bcl-2 engineered MSCs inhibited apoptosis and improved heart function. Stem Cells, 2007. 25(8): p. 2118-27.

43. Tsubokawa, T., et al., Impact of anti-apoptotic and anti-oxidative effects of bone marrow mesenchymal stem cells with transient overexpression of heme oxygenase-1 on myocardial ischemia. Am J Physiol Heart Circ Physiol, 2010. 298(5): p. H1320-9.

44. Zhou, H., et al., Exendin-4 protects adipose-derived mesenchymal stem cells from apoptosis induced by hydrogen peroxide through the PI3K/Akt-Sfrp2 pathways. Free Radic Biol Med, 2014. 77: p. 363-75.

45. Le Blanc, K., et al., Treatment of severe acute graft-versus-host disease with third party haploidentical mesenchymal stem cells. Lancet, 2004. 363(9419): p. 1439-41.

46. Lazarus, H. M., et al., Cotransplantation of HLA-identical sibling culture-expanded mesenchymal stem cells and hematopoietic stem cells in hematologic malignancy patients. Biol Blood Marrow Transplant, 2005. 11(5): p. 389-98.

47. Bernardo, M. E., et al., Optimization of in vitro expansion of human multipotent mesenchymal stromal cells for cell-therapy approaches: further insights in the search for a fetal calf serum substitute. J Cell Physiol, 2007. 211(1): p. 121-30.

48. Reinisch, A., et al., Humanized system to propagate cord blood-derived multipotent mesenchymal stromal cells for clinical application. Regen Med, 2007. 2(4): p. 371-82.

49. Capelli, C., et al., Human platelet lysate allows expansion and clinical grade production of mesenchymal stromal cells from small samples of bone marrow aspirates or marrow filter washouts. Bone Marrow Transplant, 2007. 40(8): p. 785-91.

50. Lataillade, J. J., et al., New approach to radiation burn treatment by dosimetry-guided surgery combined with autologous mesenchymal stem cell therapy. Regen Med, 2007. 2(5): p. 785-94.

51. Seshareddy, K., D. Troyer, and M. L. Weiss, Method to isolate mesenchymal-like cells from Wharton's Jelly of umbilical cord. Methods Cell Biol, 2008. 86: p. 101-19.

52. Sensebe, L., Clinical grade production of mesenchymal stem cells. Biomed Mater Eng, 2008. 18(1 Suppl): p. S3-10.

53. Sotiropoulou, P. A., S. A. Perez, and M. Papamichail, Clinical grade expansion of human bone marrow mesenchymal stem cells. Methods Mol Biol, 2007. 407: p. 245-63.

54. Shetty, P., et al., Clinical grade mesenchymal stem cells transdifferentiated under xenofree conditions alleviates motor deficiencies in a rat model of Parkinson's disease. Cell Biol Int, 2009. 33(8): p. 830-8.

55. Zhang, X., et al., Cotransplantation of HLA-identical mesenchymal stem cells and hematopoietic stem cells in Chinese patients with hematologic diseases. Int J Lab Hematol, 2010. 32(2): p. 256-64.

56. Arrigoni, E., et al., Isolation, characterization and osteogenic differentiation of adipose-derived stem cells: from small to large animal models. Cell Tissue Res, 2009. 338(3): p. 401-11.

57. Grisendi, G., et al., GMP-manufactured density gradient media for optimized mesenchymal stromal/stem cell isolation and expansion. Cytotherapy, 2010. 12(4): p. 466-77.

58. Prasad, V. K., et al., Efficacy and safety of ex vivo cultured adult human mesenchymal stem cells (Prochymal) in pediatric patients with severe refractory acute graft-versus-host disease in a compassionate use study. Biol Blood Marrow Transplant, 2011. 17(4): p. 534-41.

59. Sensebe, L., P. Bourin, and K. Tarte, Good manufacturing practices production of mesenchymal stem/stromal cells. Hum Gene Ther, 2011. 22(1): p. 19-26.

60. Capelli, C., et al., Minimally manipulated whole human umbilical cord is a rich source of clinical-grade human mesenchymal stromal cells expanded in human platelet lysate. Cytotherapy, 2011. 13(7): p. 786-801.

61. Ilic, N., et al., Manufacture of clinical grade human placenta-derived multipotent mesenchymal stromal cells. Methods Mol Biol, 2011. 698: p. 89-106.

62. Santos, F., et al., Toward a clinical-grade expansion of mesenchymal stem cells from human sources: a microcarrier-based culture system under xeno-free conditions. Tissue Eng Part C Methods, 2011. 17(12): p. 1201-10.

63. Timmins, N. E., et al., Closed system isolation and scalable expansion of human placental mesenchymal stem cells. Biotechnol Bioeng, 2012. 109(7): p. 1817-26.

64. Warnke, P. H., et al., A clinically-feasible protocol for using human platelet lysate and mesenchymal stem cells in regenerative therapies. J Craniomaxillofac Surg, 2013. 41(2): p. 153-61.

65. Fekete, N., et al., GMP-compliant isolation and large-scale expansion of bone marrow-derived MSC. PLoS One, 2012. 7(8): p. e43255.

66. Hanley, P. J., et al., Manufacturing mesenchymal stromal cells for phase I clinical trials. Cytotherapy, 2013. 15(4): p. 416-22.

67. Trojahn Kolle, S. F., et al., Pooled human platelet lysate versus fetal bovine serum-investigating the proliferation rate, chromosome stability and angiogenic potential of human adipose tissue-derived stem cells intended for clinical use. Cytotherapy, 2013. 15(9): p. 1086-97.

68. Veronesi, E., et al., Transportation conditions for prompt use of ex vivo expanded and freshly harvested clinical-grade bone marrow mesenchymal stromal/stem cells for bone regeneration. Tissue Eng Part C Methods, 2014. 20(3): p. 239-51.

69. Dolley-Sonneville, P. J., L. E. Romeo, and Z. K. Melkoumian, Synthetic surface for expansion of human mesenchymal stem cells in xeno-free, chemically defined culture conditions. PLoS One, 2013. 8(8): p. e70263.

70. Siciliano, C., et al., Optimization of the isolation and expansion method of human mediastinal-adipose tissue derived mesenchymal stem cells with virally inactivated GMP-grade platelet lysate. Cytotechnology, 2015. 67(1): p. 165-74.

71. Martins, J. P., et al., Towards an advanced therapy medicinal product based on mesenchymal stromal cells isolated from the umbilical cord tissue: quality and safety data. Stem Cell Res Ther, 2014. 5(1): p. 9.

72. Iudicone, P., et al., Pathogen-free, plasma-poor platelet lysate and expansion of human mesenchymal stem cells. J Transl Med, 2014. 12: p. 28.

73. Skrahin, A., et al., Autologous mesenchymal stromal cell infusion as adjunct treatment in patients with multidrug and extensively drug-resistant tuberculosis: an open-label phase 1 safety trial. Lancet Respir Med, 2014. 2(2): p. 108-22.

74. Ikebe, C. and K. Suzuki, Mesenchymal stem cells for regenerative therapy: optimization of cell preparation protocols. Biomed Res Int, 2014. 2014: p. 951512.

75. Chatzistamatiou, T. K., et al., Optimizing isolation culture and freezing methods to preserve Wharton's jelly's mesenchymal stem cell (MSC) properties: an MSC banking protocol validation for the Hellenic Cord Blood Bank. Transfusion, 2014. 54(12): p. 3108-20.

76. Swamynathan, P., et al., Are serum-free and xeno-free culture conditions ideal for large scale clinical grade expansion of Wharton's jelly derived mesenchymal stem cells? A comparative study. Stem Cell Res Ther, 2014. 5(4): p. 88.

77. Vaes, B., et al., Culturing protocols for human multipotent adult stem cells. Methods Mol Biol, 2015. 1235: p. 49-58.

78. Devito, L., et al., Wharton's jelly mesenchymal stromal/stem cells derived under chemically defined animal product-free low oxygen conditions are rich in MSCA-1(+) subpopulation. Regen Med, 2014. 9(6): p. 723-32.

1. A system for treating a subject comprising:
a. at least one bioreactor comprising cells,
b. at least one selectively permeable membrane, and
c. tubing connecting at least one of the bioreactor(s), at least one of the selectively permeable membrane(s), and the subject in order to circulate fluid from the subject to the bioreactor, with the proviso that if the tubing comprises a hollow fiber then a membrane is optional.

2. The system of claim 1, further comprising blood or plasma from the subject or comprising organ preservation solution.

3. The system of claim 1, wherein the cells are regenerative cells.

4. The system of claim 3, wherein the regenerative cells are fibroblasts, dedifferentiated fibroblasts, inducible pluripotent cells, parthenogenic derived cells, mesenchymal stem cells, or hematopoietic stem cells.

5. The system of claim 4, wherein the regenerative cells secrete one or more regenerative factors at a basal or at an inducible rate.

6. The system of claim 5, wherein the regenerative factors include one or more factors selected from the group consisting of AKT, BAMBI, BCL-2, BCL-2XL, BDNF, BIRC5 CDA, CXCR4, dominant negative CCL2, EGF, exosomes, FGF-2, GATA-4 GDF-11, GDNF, hCG, HGF, HIF-1alpha, HLA-G, HO-1, hTERT, IFN-b, IGF-1, IFT-1, LIGHT, miR-126, NK4, NUR77, OCT-4, PGE-1, SDF-1, STC-1, TERT, TRAIL, VEGF, WNT11, XIAP, and a combination thereof.

7. The system of claim 4, wherein the regenerative cells produce one or more regenerative factors in response to degenerative factors from the blood or plasma of the subject.

8. The system of claim 4, wherein the regenerative cells secrete regenerative factors at a rate set by the detection of degenerative factors.

9. The system of claim 8, wherein the rate of regenerative factor secretion from the cells is a ratio to the rate of degenerative factor detection by the cells.

10. The system of claim 9, wherein the ratio of regenerative factor secretion rate to degenerative factor detection rate is selected from the group consisting of 50:1, 10:1, 5:1, 1:1, 1:5, 1:10, 1:50, and any ratio between.

11. The system of claim 1 wherein a selectively permeable membrane is positioned in the tubing between the bioreactor and the subject, the tubing is comprised of a selectively permeable membrane, and/or the bioreactor is comprised, at least in part, of a selectively permeable membrane.

12. The system of claim 11, wherein the selectively permeable membrane inhibits or reduces the passage of cellular material between the subject and a bioreactor.

13. The system of claim 1, wherein one or more selectively permeable membrane(s) permits the passage of regenerative and degenerative factors.

14. The system of claim 1, wherein the subject is an animal.

15. The system of claim 1, wherein the subject is an organ or tissue from an animal.

16. The system of claim 1, wherein the subject is an organ or tissue from a donor.

17. The system of claim 15, wherein the organ is selected from the group consisting of liver, pancreas, gallbladder, stomach, small intestine, large intestine, lung, kidney, heart, spleen, brain, eye, and a combination thereof.

18. The system of claim 15, wherein the subject is any part of the organ or any part of an organ system.

19. The system of claim 1, wherein the subject is an organ or tissue and the system also comprises a container connected to at least one of the bioreactor(s) and at least one of the selectively permeable membrane(s) for encapsulating the subject.

20. The system of claim 19, wherein the subject is an organ or tissue and the container is suitable for holding the subject to allow for transfer of organ preservation or other fluid into the tubing.

21. An extracorporeal method of producing regenerative factors, comprising the step of subjecting a subject to the system of claim 1 under conditions that allow for secretion of one or more regenerative factors from the cells in the system.

22. The method of claim 21, wherein the cells are fibroblasts that are derived from tissues comprising skin, heart, blood vessels, bone marrow, skeletal muscle, liver, pancreas, brain, adipose tissue, placenta, and/or foreskin.

23. The method of claim 22, wherein the fibroblasts have one or more surface markers selected from the group consisting of CD73, CD90, CD56, SSEA3, SSEA4, Tra-1-60, Tra-1-81, Tra-2-54, HLA class I, CD13, CD44, CD49b, CD105, aminopeptidase N, hyaluronic acid-binding receptor, collagen/laminin-binding integrin alpha 2, OCT4, NANOG, SOX-2, and a combination thereof.

24. The method of claim 20, wherein the fibroblasts lack one or more surface markers selected from the group consisting of CD14, CD34, CD45, HLA Class II, and a combination thereof.

25. The method of claim 22, wherein the method comprises the step(s) of:
a. culturing fibroblasts in an undifferentiated state for 1, 2, 3, 4, 5, 6, 7, 8, 9, 10, 11, 12, 13, 14, 15, 16, 17, 18, 19, 20, 21, 22, 23, 24, 25 or more days;
b. culturing the fibroblasts from step (a) in the presence of one or more factors selected from the group consisting of nerve growth factor, bFGF, dibutryl cAMP, IBMX, retinoic acid, exendin-4, and a combination thereof; and
c. activating the fibroblasts.

26. The method of claim 25, wherein activating the fibroblasts comprises exposing the fibroblasts to one or more cytokines in cell culture media.

27. The method of claim 26, wherein the cytokine is selected from the group consisting of IL-1, IFNgamma, and a combination thereof.

28. The method of claim 27, wherein the concentration of IL-1 is 1-100 ng/mL, 5-100 ng/mL, 10-100 ng/mL, or 20-40 ng/mL.

29. The method of claim 27, wherein the concentration of IL-1 is 1 ng/mL, 5 ng/mL, 10 ng/mL, 20 ng/mL, 30 ng/mL, 40 ng/mL, 50 ng/mL, 60 ng/mL, 70 ng/mL, 80 ng/mL, 90 ng/mL, or 100 ng/mL.

30. The method of claim 27, wherein the concentration of IFNgamma is 1-1000 IU, 5-1000 IU, 10-1000 IU, 1-500 IU, 5-500 IU, 10-500 IU, 100-500 IU, or 250 IU.

31. The method of claim 27, wherein the concentration IFNgamma is 1 IU, 5 IU, 10 IU, 50 IU, 100 IU, 200 IU, 250 IU, 300 IU, 400 IU, 500 IU, 600 IU, 700 IU, 800 IU, 900 IU, or 1000 IU.

32. The method of claim 22, wherein exposing the fibroblasts to one or more cytokines induces an increase in the expression of one or more complement inhibitory molecules from the fibroblasts.

33. The method of claim 32, wherein the complement inhibitory molecules are selected from the group consisting of CD35, CD46, C4BP, CD55, Factor H, and a combination thereof.

34. The method of claim 25, wherein activating the fibroblasts comprises transfecting the fibroblasts with one or more viral and/or non-viral expression systems to induce the expression of one or more regenerative factors.

35. The method of claim 34, wherein the regenerative factors are selected from the group consisting of AKT, BAMBI, BCL-2, BCL-2XL, BDN, BIRC5 CDA, CXCR4, dominant negative CCL2, EGF, exosomes, FGF-2, GATA-4 GDF-11, GDNF, hCG, HGF, HIF-1alpha, HLA-G, HO-1, hTERT, IFN-b, IFT-1, LIGHT, miR-126, NK4, NUR77, OCT-4, PGE-1, SDF-1, STC-1, TERT, TRAIL, VEGF, WNT11, XIAP, and a combination thereof.

36. A kit comprising the system of claim 1.
